(19) United States
(12) Patent Application Publication
(10) Pub. No.: US 2020/0095566 A1
(43) Pub. Date: Mar. 26, 2020

(54) Protein Expression Construct and Methods Thereof

(71) Applicant: AGENCY FOR SCIENCE, TECHNOLOGY AND RESEARCH, Singapore (SG)

(72) Inventors: Dave Siak Wei OW, Singapore (SG); Fong Tian WONG, Singapore (SG)

(21) Appl. No.: 16/495,987

(22) PCT Filed: Mar. 8, 2018
(86) PCT No.: PCT/SG2018/050107

§ 371 Date: Sep. 20, 2019

(30) Foreign Application Priority Data
Mar. 22, 2017 (SG) 10201702333U

(51) Int. Cl. C12N 9/64; A61K 35/747; A61K 35/744

(52) U.S. Cl. CPC C12N 9/6424; C12Y 304/21026; A61K 38/00; A61K 35/744; A61K 35/747

(57) ABSTRACT

The invention relates generally to the field of microbiology and molecular biology. Provided herein is an expression construct for producing a recombinant protein. The subject specification discloses an expression construct for producing a prolyl endopeptidase protein in lactic acid bacteria and methods of treatment comprising use of such a prolyl endopeptidase.

### FIELD

The invention relates generally to the field of microbiology and molecular biology. Provided herein is an expression construct for producing a recombinant protein. The subject specification discloses an expression construct for producing a prolyl endopeptidase protein in lactic acid bacteria and methods of treatment comprising use of such a prolyl endopeptidase.

### BACKGROUND

Bibliographic details of the publications referred to by author in this specification are collected alphabetically at the end of the description.

The reference in this specification to any prior publication (or information derived from it), or to any matter which is known, is not, and should not be taken as an acknowledgement or admission or any form of suggestion that the prior publication (or information derived from it) or known matter forms part of the common general knowledge in the field of endeavor to which this specification relates.

Celiac disease is an inflammatory autoimmune disorder of the small intestine arising from intolerance to gluten (a protein found in wheat, rye, barley and oats) in food. Typically, the tiny finger-like projections which line the bowel becomes inflamed and flattened leading to villous atrophy. Symptoms of the disease include gastrointestinal problems such as chronic diarrhoea, malabsorption or loss of appetite.

Traditional treatment of celiac disease mainly consists of dietary restrictions, however even traces of gluten contaminants can be immunogenic and result in detrimental consequences over time. Results from development of an oral therapy for Celiac disease using food grade prolyl endopeptidase (PEP) to break down contaminant gluten have been promising. There are also ongoing studies to develop non-dietary alternatives using mucosal enzyme vectors to deliver digestive enzymes to patients.

Lactic acid bacteria (LAB) are a promising family of food-grade organisms for heterologous protein production due to its Generally Regarded as Safe (GRAS) status. Traditionally, LAB was utilized in food as starter cultures for fermentation and as probiotics. Studies of LAB and host interactions have also associated LAB directly with cellular activities of the gut, such as pathogen control, immune-stimulation and maintaining a healthy microflora. Combined with their traditional roles in food fermentation, beneficial gut properties, and resistance to harsh gut conditions, additional advantages such as LAB's ability to secrete recombinant proteins while possessing fewer proteases, compared to traditional workhorses, such as Escherichia coli and Bacillus subtilis, have made them attractive targets as recombinant cell factories and live vectors for the delivery of therapeutic molecules to the gut.

### SUMMARY

The present specification discloses an expression construct for producing a protein in lactic acid bacteria. In one aspect, the invention provides tools for efficient expression of recombinant protein in lactic acid bacteria. In another aspect, the invention provides better means to deliver digestive enzymes to a patient for the treatment of Celiac disease. Provided herein is an expression construct encoding a fusion protein, wherein the construct comprises a) a first nucleic acid sequence encoding a peptide of general formula (I):

(SEQ ID NO 1)
X₁-X₂-X₃-X₄-X₅-X₆-X₇-X₈-X₉-X₁₀

wherein each of X₁, X₅ and X₁₀ is a negatively charged amino acid or a functional variant thereof, wherein each of X₂, X₃, X₄, X₆, X₇ and X₉ is a polar or non-polar amino acid, or a functional variant thereof, wherein X₈ is a positively-charged or a polar amino acid, or a functional variant thereof; and b) a second nucleic acid sequence encoding a protein for expression; wherein the first nucleic acid sequence is contiguous to the second nucleic acid sequence, such that the peptide encoded by the first nucleic acid sequence and the protein encoded by the second nucleic acid sequence forms a fusion protein; wherein the peptide encoded by the first nucleic acid sequence improves the expression of the protein encoded by the second nucleic acid sequence as compared to when the peptide encoded by the first nucleic acid sequence is absent.

Provided herein is a method of expressing a protein in a lactic acid bacterium, the method comprising the steps of culturing the recombinant lactic acid bacterium as defined herein and isolating the protein expressed by the bacterium.

Provided herein is a recombinant lactic acid bacterium comprising an expression construct as defined herein.

Provided herein is a method of treating a gut disease, the method comprising the step of administering a recombinant lactic acid bacterium as defined herein or a protein expressed by the method as defined herein to a patient in need thereof.

Provided herein is a recombinant lactic acid bacterium as defined herein or a protein expressed by the method as defined herein for use in treating a gut disease.

Provided herein is the use of a recombinant lactic acid bacterium as defined herein or a protein expressed by the method as defined herein in the manufacture of a medicament for the treatment of a gut disease.

In one embodiment, the gut disease is a Celiac disease.

### BRIEF DESCRIPTION OF THE FIGURES

FIG. 1: Mining of propeptides from sequenced Lactococcus genome assemblies. FIG. 1(a) shows a schematic representation of the alignment of native Lactococcus proteins with USP45-LEISSTCDA (SEQ ID NO: 2) sequence. FIG. 1(b) shows a schematic representation of the isoelectric points and net charge of the three propeptides (PP1-PP3) and positive control LEISSTCDA (PC) propeptide.

FIG. 2: Secretion of TRX in NZ9000. FIG. 2(a) shows a schematic representation of the expression construct for secreted TRX, USP: USP45, PP: Propeptide, Linker: Flexible linker with TEV cleavage site, His6: HisTag, Term: Terminator. FIG. 2(b) shows a graphical representation of growth curves for pNZ8148 vector only, USP45-TRX, USP45-PP1-PP2-, -PP3-, -PC-TRX, FIG. 2(c) shows a photographic representation of the comparison of cell lysate (C) and secreted (S) fractions (% yields and efficiencies are given below the figure). These are calculated based on densitometry measurements of the bands.

FIG. 3: Secretion of TRX in NZ9000. FIG. 3(a) shows a schematic representation of the expression construct for secreted TRX, USP: USP45, PP: Propeptide, Linker: Flexible linker with TEV cleavage site. His6: HisTag, Term: Terminator. FIG. 3(b) shows a graphical representation of growth curves for pNZ8148 vector only, USP45-TRX, USP45-PP1-PP2-, -PP3-, -PC-TRX, Induction is indicated by a arrow. FIG. 3(c) shows a photographic representation of a representative Western blot of cell lysate (C) and secreted (S) fractions. Cell lysate and secreted fractions were concentrated 3 and 25 times respectively and 2 μL of each was loaded onto the gel. The lowest conserved band observed in the cell lysate fraction is a non-specific binding artefact (FIG. 6). % protein yields, with reference to USP45-TRX, and secretion efficiencies of the various TRX constructs are given below. These were calculated based on densitometry and technical triplicates.

FIG. 4: Secretion of Fm PEP in NZ9000. FIG. 4(a) shows a schematic representation of the expression construct for secreted FmPEP, USP: USP45, PP: Propeptide, His6: HisTag, Term: Terminator, FIG. 4(b) shows a graphical representation of growth curves for pNZ8148 vector only, USP45-FmPEP, USP45-PP1-, -PP2-, -PP3-, -PC-FmPEP. FIG. 4(c) shows a photographic representation of the comparison of media fractions. Vector refers to expression of an empty pNZ8148 vector. FIG. 4(d) shows a photographic representation of the comparison of soluble (S) and insoluble (IS) fractions in the cell lysate. Vector refers to expression of an empty pNZ8148 vector. FIG. 4(e) shows a graphic representation of the % secretion yields and secretion efficiencies for the various constructs with respect to USP45-FmPEP. Secretion protein yields are calculated based on densitometry. Enzyme activities were calculated based on Z-gly-pro-4-nitroanilide assay. Biological triplicates were performed for these experiments.

FIG. 5: Secretion of Fm PEP in NZ9000. FIG. 5(a) shows a schematic representation of the expression construct for secreted Fm PEP, USP: USP45, PP: Propeptide, His6: HisTag, Term: Terminator. FIG. 5(b) shows a graphical representation of growth curves for pNZ8148 vector only, USP45-FmPEP, USP45-PP1-, -PP2-, -PP3-, -PC-FmPEP. Induction is indicated by a red arrow. FIG. 5(c) shows a photographic representation of the comparison of media fractions on a representative Western blot. Vector refers to expression of an empty pNZ8148 vector. FIG. 5(d) shows a photographic representation of a representative Western blot comparison of soluble (S) and insoluble (IS) fractions in the cell lysate. Vector refers to expression of an empty pNZ8148 vector. Cell lysate and secreted fractions were concentrated 3 and 25 times respectively and 2 μL of each was loaded onto the gel. FIG. 5(e) shows a graphical representation of the % secretion yields and secretion efficiencies for the various constructs with respect to USP45-Fm PEP. Secretion protein yields are calculated based on densitometry. Enzyme activities were calculated based on Z-gly-pro-4-nitroanilide assay, Biological and technical triplicates were performed for these experiments, significant at *p<0.05, **p<0.01. Results are summarized in Table 1.

FIG. 6: TRX secretion. FIG. 6 shows a photographic representation of cell lysate (C) and secreted (S) fractions for NZ9000 strains containing vector* (empty pNZ8148 only), constructs with TRX without USP45 SP and USP45 SP-TRX.

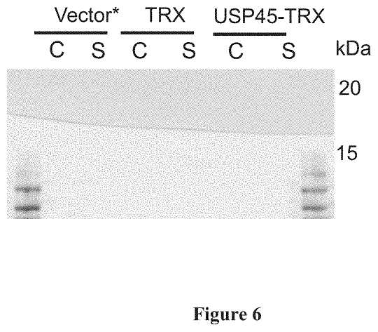

FIG. 7 is a graphical representation of the enzymatic activity of a representative set of intracellular fractions for pNZ8148 vector only, USP45-FmPEP, USP45-PP1-, -PP2-. -PP3- and -PC-FmPEP. Release of p-nitroanilide, by cleavage of Z-gly-pro-4-nitroanilide, was measured at 410 nm with time (seconds).

### DETAILED DESCRIPTION

Throughout this specification, unless the context requires otherwise, the word “comprise”, or variations such as “comprises” or “comprising”, will be understood to imply the inclusion of a stated element or integer or method step or group of elements or integers or method steps but not the exclusion of any other element or integer or method steps or group of elements or integers or method steps.

As used in the subject specification, the singular forms “a”, “an” and “the” include plural aspects unless the context clearly dictates otherwise. Thus, for example, reference to “a method” includes a single method, as well as two or more methods; reference to “an agent” includes a single agent, as well as two or more agents; reference to “the disclosure” includes a single and multiple aspects taught by the disclosure; and so forth. Aspects taught and enabled herein are encompassed by the term “invention”. Any variants and derivatives contemplated herein are encompassed by “forms” of the invention.

The following abbreviations are used throughout the application: GRAS=generally regarded as safe; SP=signal peptide; PC=positive control; TRX=Thioredoxin; Fm PEP=Flavobacterium meningosepticum prolyl endopeptidase; and Mx PEP=Myxococcus xanthus prolyl endopeptidase.

The present specification discloses an expression construct for producing a protein in lactic acid bacteria. Provided herein is an expression construct encoding a fusion protein, wherein the construct comprises a) a first nucleic acid sequence encoding a peptide of general formula (I):

(SEQ ID NO 1)
X₁-X₂-X₃-X₄-X₅-X₆-X₇-X₈-X₉-X₁₀.

The term “expression construct” may refer to a nucleic acid molecule containing a desired coding sequence and appropriate nucleic acid sequences necessary for expression of the operably linked coding sequence (e.g. an insert sequence that codes for a product) in a particular host cell. Nucleic acid sequences necessary for expression in prokaryotes usually include a promoter and a ribosome binding site, often along with other sequences. In one example, the expression construct is suitable for expression in lactic acid bacteria.

The term “encode” or “encoding” includes reference to nucleotides and/or amino acids that correspond to other nucleotides or amino acids in the transcriptional and/or translational sense.

The terms “protein” and “polypeptide” are used interchangeably and refer to any polymer of amino acids (dipeptide or greater) linked through peptide bonds or modified peptide bonds. Polypeptides of less than about 10-20 amino acid residues are commonly referred to as “peptides.” The polypeptides of the invention may comprise non-peptidic components, such as carbohydrate groups. Carbohydrates and other non-peptidic substituents may be added to a polypeptide by the cell in which the polypeptide is produced, and will vary with the type of cell. Polypeptides are defined herein, in terms of their amino acid backbone structures; substituents such as carbohydrate groups are generally not specified, but may be present nonetheless.

The term “nucleic acid” includes a deoxyribonucleotide or ribonucleotide polymer in either single- or double-stranded form, and unless otherwise limited, encompasses known analogues of natural nucleotides that hybridize to nucleic acids in a manner similar to naturally occurring nucleotides. The terms “nucleic acid”, “nucleic acid molecule”, “nucleic acid sequence” and “polynucleotide” are used interchangeably herein unless the context indicates otherwise.

The terms “non-polar amino acids”, “polar amino acids”, “hydrophobic amino acids”, “positively charged amino acids” “negatively charged amino acids” are all used consistently with the prior art terminology. Each of these terms is well-known in the art and has been extensively described in numerous publications, including standard biochemistry text books, describing properties of amino acids which lead to their definition as polar, non-polar or acidic.

In general, the non-polar amino acids may refer to glycine, alanine, valine, isoleucine, leucine and proline. The non-polar amino acids may also include aromatic non-polar amino acids such as phenylalanine, tryptophan and tyrosine. The neutral polar amino acids may refer to serine, threonine, cysteine, glutamine, asparagine and methionine. The negatively charged amino acids may refer to aspartic acid and glutamic acid. The positively charged amino acids may refer to histidine or arginine.

In one example, each of X₁, X₅ and X₁₀ is a negatively charged amino acid or a functional variant thereof.

In one example, each of X₂, X₃, X₄, X₆, X₇ and X₉ is a polar or non-polar amino acid, or a. functional variant thereof.

In one example, X₈ is a positively-charged or a polar amino acid, or a functional variant thereof.

The term “functional variant” may refer to natural or chemically synthesized derivatives or analogues of an amino acid that is known to a person skilled in the art. A “functional variant” of an amino acid may have one or more modifica.tion(s) or variation(s) to its side chain moieties. For example, a side chain moiety of a D- or L-amino acid may have been modified to include a straight chain or branched, cyclic or non-cyclic, substituted or non-substituted, saturated or unsaturated, alkyl, aryl or aralyl moiety. A side chain of a D- or L-amino acid may have been modified to include reactive functional groups such as an azido or alkyne group. The term “functional variant” may also include, but is not limited to, amino acids that have been modified by addition of one or more sugar/carbohydrate moiety, oligosaccharide, or lipid groups. A “functional variant” may be incorporated in vivo into a recombinant protein via techniques that are known in the art. For example, a “functional variant” of an amino acid may be incorporated into a recombinant protein via selective pressure incorporation in bacteria. Orthogonal aminoacyl-tRNA synthetase and tRNA may also be used to direct incorporation of a “functional variant” of an amino acid into a recombinant protein in bacteria in response to a codon on the expression construct.

The construct may comprise b) a second nucleic acid sequence encoding a protein for expression.

The first nucleic acid sequence may be contiguous to the second nucleic acid sequence, such that the peptide encoded by the first nucleic acid sequence and the protein encoded by the second nucleic acid sequence forms a fusion protein.

The term “fusion protein” refers to a chimera of at least two covalently bonded polypeptide molecules.

The term “contiguous” refers to two nucleic acids being adjacent to one another. The two nucleic acid molecules may be in the same reading frame that allows the formation of a “fusion protein”. In one example, the peptide encoded by the first nucleic acid sequence is positioned at the N terminus end of the protein encoded by the second nucleic acid sequence.

In one example, the peptide encoded by the first nucleic acid sequence improves the expression of the protein encoded by the second nucleic acid sequence as compared to when the peptide encoded by the first nucleic acid sequence is absent. In one example, the improvement of expression is in terms of the overall yield of protein produced by the bacteria. In one example, the improvement of expression is in terms of the volumetric protein yield of the bacteria. In one example, the improvement of expression is in terms of the specific protein yield of the bacteria. In one example, the improvement of expression is in terms of the amount of enzymatically active protein that is produced. In another example, the improvement of expression is in terms of the secretion efficiency of the bacteria.

The peptide encoded by the first nucleic acid may be modified to have insertions, deletions or substitutions, either conservative or non-conservative, provided that such changes allow the modified peptide to retain the activity of the original peptide (i.e. improving the expression of the protein encoded by the second nucleic acid). Each of these types of changes may occur alone, or in combination with the others, one or more times in a given sequence. Such changes may, for example, be made using the methods of protein engineering and site-directed mutagenesis. A “conservative” change is wherein a substituted amino acid has similar structural or chemical properties. A “non-conservative” change is wherein the substituted amino acid is structurally or chemically different.

In one example, there is provided an expression construct encoding a fusion protein, wherein the construct comprises a) a first nucleic acid sequence encoding a peptide of general formula (I):

(SEQ ID NO 1)
X₁-X₂-X₃-X₄-X₅-X₆-X₇-X₈-X₉-X₁₀.

wherein each of X₁, X₅ and X₁₀ is a negatively charged amino acid or a functional variant thereof, wherein each of X₂, X₃, X₄, X₆, X₇ and X₉ is a polar or non-polar amino acid, or a functional variant thereof, wherein X₈ is a positively-charged or a polar amino acid, or a functional variant thereof; and b) a second nucleic acid sequence encoding a protein for expression; wherein the first nucleic acid sequence is contiguous to the second nucleic acid sequence, such that the peptide encoded by the first nucleic acid sequence and the protein encoded by the second nucleic acid sequence forms a fusion protein; wherein the peptide encoded by the first nucleic acid sequence improves the expression of the protein encoded by the second nucleic acid sequence as compared to when the peptide encoded by the first nucleic acid sequence is absent.

In one example, each of X₁, and X₅ is an aspartic acid, or a functional variant thereof. In one example, X₂ is a threonine or a functional variant thereof. In one example, X₃ is selected from the group consisting of asparagine, threonine, and serine, or a functional variant thereof. In one example, X₄ is selected from the group consisting of serine, threonine and alanine, or a functional variant thereof. In one example, X₆ is an isoleucine, or a functional variant thereof. In one example, X₇ is an alanine or a functional variant thereof. In one example, X₈ is selected from the group consisting of lysine and asparagine, or a functional variant thereof In one example, X₉ is a glutamine or a functional variant thereof. In one example, X₁₀ is selected from the group consisting of aspartic acid and glutamic acid or a functional variant thereof.

In one example, X₁ is optionally present or absent. In another example, X₁₀ is optionally present or absent.

The peptide encoded by the first nucleic acid sequence may be of the general formula: D-X₂-X₃-X₄-X₅-X₆-X₇-X₈-X₉-X₁₀ (SEQ ID NO: 3), X₁-T-X₃-X₄-X₅-X₆-X₇-X₈-X₉-X₁₀ (SEQ ID NO: 4), X₁-X₂-X₃-X₄-D-X₆-X₇-X₈-X₉-X₁₀ (SEQ ID NO: 5), X₁-X₂-X₃-X₄-X₅-I-X₇-X₈-X₉-X₁₀ (SEQ ID NO: 6), X₁-X₂-X₃-X₄-X₅-X₆-A-X₈-X₉-X₁₀ (SEQ ID NO:7) or X₁-X₂-X₃-X₄-X₅-X₆-X₇-X₈-Q-X₁₀ (SEQ ID NO: 8).

The peptide encoded by the first nucleic acid sequence may be of the general formula: D-T-X₃-X₁-X₅-X₆-X₇-X₈-X₉-X₁₀ (SEQ ID NO: 9), D-X₂-X₃-X₄-D-X₆-X₇-X₈-X₉-X₁₀(SEQ ID NO: 10), D-X₂-X₃-X₄-X₅-I-X₇-X₈-X₉-X₁₀ (SEQ ID NO: 11). D-X₂-X₃-X₄-X₅-X₆-A-X₈-X₉-X₁₀ (SEQ ID NO: 12), D-X₂-X₃-X₄-X₅-X₆-X₇-X₈-Q-X₁₀ (SEQ ID NO: 13), X₁-T-X₃-X₄-D-X₆-X₇-X₈ X₉-X₁₀ (SEQ ID NO: 14), X₁-T-X₃-X₄-X₅-I-X₇-X₈-X₉-X₁₀ (SEQ ID NO: 15), X₁-T-X₃-X₄-X₅-X₆-A-X₈-X₉-X₁₀ (SEQ ID NO: 16), X₁-T-X₃-X₄-X₅-X₆-X-₇-X₈-Q-X₁₀ (SEQ ID NO: 17), X₁-X₂-X₃-X₄-D-I-X₇-X₈-X₉-X₁₀ (SEQ ID No: 18), X₁-X₂-X₃-X₄-D-X₆-A-X₈-X₉-X₁₀ (SEQ ID NO: 19), X₁-X₂-X₃-X₄-D-X₆-X₇-X₈-Q-X₁₀ (SEQ ID NO: 20), X₁-X₂-X₃-X₄-X₅-I-A-X₈-X₉-X₁₀ (SEQ ID NO: 21), X₁-X₂-X₃-X₄-X₅-I-X₇-X₈-Q-X₁₀ (SEQ ID NO: 22) and X₁-X₂-X₃-X₄-X₅-X₆-A-X₈-Q-X₁₀ (SEQ ID NO: 23).

In one example, the peptide encoded by the first nucleic acid sequence is D-T-X₃-X₄-D-I-A-X₈-Q-X₁₀ (SEQ ID NO: 24). X₃ and X₄ may each be a polar or non-polar amino acid, or a functional variant thereof. X₈ may be a positively-charged or a polar amino acid, or a functional variant thereof. X₁₀ may be a negatively charged amino acid or a functional variant thereof.

In one example, the peptide encoded by the first nucleic acid sequence is of general formula (1a): D-T-X₃-X₄-D-I-A-X₈-Q-X₁₀ (SEQ ID NO: 25).

In one example, X₃ is selected from the group consisting of asparagine, threonine, and serine, or a functional variant thereof. In one example, X₄ is selected from the group consisting of serine, threonine and alanine, or a functional variant thereof. In one example, X₈ is selected from the group consisting of lysine and asparagine, or a functional variant thereof. In one example, X₁₀ is selected from the group consisting of an aspartic acid and a glutamic acid, or a functional variant thereof.

The peptide encoded by the first nucleic acid sequence may be at least 60%, 70%, 80% or 90° identical to a sequence selected from the group consisting of: a) DTNSDIAKQD (SEQ ID NO: 26); b) DTTTDIAKQE (SEQ ID NO: 27); and c) DTSADIANQE (SEQ ID NO: 28). In one example, the peptide encoded by the first nucleic acid sequence is at least 90% identical to a sequence selected from the group consisting of: a) DTNSDIAKQD (SEQ ID NO: 26); b) DTTTDIAKQE (SEQ ID NO: 27); and c) DTSADIANQE (SEQ ID NO: 28). In one example, the peptide encoded by the first nucleic acid sequence is DTNSDIAKQD (SEQ NO: 26). In one example, the peptide encoded by the first nucleic acid sequence is DTTTDIAKQE (SEQ ID NO: 27). In one example, the peptide encoded by the first nucleic acid sequence is DTSADIANQE (SEQ ID NO: 28).

The term “sequence identity” as used herein refers to the extent that sequences are identical on an amino acid-by-amino acid basis over a window of comparison. Thus, a “percentage of sequence identity” is calculated by comparing two optimally aligned sequences over the window of comparison, determining the number of positions at which the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. Methods of aligning amino acid sequences are well known in the art. For example, bioinformatics or computer programs and alignment algorithms such as ClusterW may be used to determine the “% identity” between two amino acid sequences.

In one example, the peptide encoded by the first nucleic acid sequence has a net negative charge of −2 to −3 at 7.

The protein encoded by the second nucleic acid sequence may be prolyl endopeptidase (PEP) or thioredoxin.

In one example, the prolyl endopeptidase (PEP) is selected from a group consisting of a Myxococeus Xanthus prolyl endopeptidase, a Flavobacterium meningosepticum prolyl endopeptidase, an Aspergillus niger prolyl endopeptidase and a Sphingomonas capsulate prolyl endopeptidase.

In one example, the peptide encoded by the first nucleic acid sequence is positioned between a signal peptide and the protein encoded by the second nucleic acid sequence. The signal peptide is typically positioned at the N-terminus of the fusion protein. In one example, the signal peptide is positioned N-terminally to the peptide encoded by the first nucleic acid, while the peptide encoded by the first nucleic acid sequence is positioned N-terminally to the protein encoded by the second nucleic acid. In one example, the signal peptide is a USP45 signal peptide. In one example, the signal peptide is cleaved off from the fusion protein upon secretion whereas the peptide encoded by the first nucleic acid sequence and the protein encoded by the second nucleic acid sequences remains as a fusion protein after secretion.

The terms “signal sequence” or “signal peptide refers to a short (about 5 to about 60 amino acids long) peptide that directs co- or post-translational transport of a protein from the cytosol to certain organelles such as the nucleus, mitochondrial matrix, and endoplasmic reticulum, for example. For proteins having an ER targeting signal peptide, the signal peptides are typically cleaved from the precursor form by signal peptidase after the proteins are transported to the ER, and the resulting proteins move along the secretory pathway to their intracellular (e.g., the Golgi apparatus, cell membrane or cell wall) or extracellular locations. “ER targeting signal peptides,” as used herein include amino-terminal hydrophobic sequences which are usually enzymatically removed following the insertion of part or all of the protein through the ER membrane into the lumen of the ER. Thus, it is known in the art that a signal precursor form of a sequence can be present as part of a precursor form of a protein, but will generally be absent from the mature form of the protein.

In one example, there is provided a recombinant lactic acid bacterium comprising an expression construct as defined herein.

The term “recombinant” includes reference to a cell that has been modified by the introduction of a heterologous nucleic acid, or a cell derived from a cell that has been modified in such a manner, but does not encompass the alteration of the cell by naturally occurring events (e.g., spontaneous mutation, natural transformation, natural transduction, natural transposition) such as those occurring without deliberate human intervention.

In one example, the genus of the lactic acid bacterium is selected from the group consisting of Lactobacillus, Lactococcus, Aerococcus, Leuconostoc, Oenococcus, Pediococcus, Streptococcus, Enterococcus, Weisella, Allolococcus, Carnobacterium, Dolosigranulum, Globicatella, Tetrogenococcus and Vagococcus.

In one example, the lactic acid bacterium is Lactococcus lactis or Lactobacillus spp.

In one example, the lactic acid bacterium is in a freeze-dried or lyophilized formulation. The bacterium may also be reconstituted for use as a medicament.

In one example, there is provided a method of expressing a protein in a lactic acid bacterium, the method comprising the steps of culturing the recombinant lactic acid bacterium as defined herein and isolating the protein expressed by the bacterium.

In one example, there is provided a recombinant lactic acid bacterium as defined herein or a protein expressed by the method as defined herein for use as a medicament.

In one example, there is provided a method of treating a gut disease, the method comprising the step of administering a recombinant lactic acid bacterium as defined herein or a protein expressed by the method as defined herein to a patient in need thereof.

In one example, there is provided a method of delivering a protein to the gut of a subject, the method comprising the step of administering a recombinant lactic acid bacterium as defined herein to the subject.

The recombinant lactic acid bacterium or protein expressed by the method as defined herein may be in the form of a solid or liquid pharmaceutical composition. Pharmaceutical compositions can be formulated with a pharmaceutically acceptable carrier for administration to a subject. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water, saline solutions and aqueous dextrose and glycerol solutions can also be employed as liquid carriers. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of recombinant lactic acid bacteria or recombinant protein together with a suitable amount of carrier so as to provide the form for proper administration to the patient. The formulation should suit the mode of administration.

The term “administering” and variations of that term including “administer” and “administration”, includes contacting, applying, delivering or providing a pharmaceutically effective amount of the recombinant lactic acid bacteria or protein to an organism, or a surface by any appropriate means. The recombinant bacteria or protein may be administered in dosages and by techniques well known to those skilled in the medical or veterinary arts, taking into consideration such factors as the age, sex, weight, species and condition of the recipient animal, and the route of administration. The route of administration can be percutaneous, via mucosal administration (e.g., oral, nasal, anal, vaginal) or via a parenteral route (intradermal, intramuscular, subcutaneous, intravenous, or intraperitoneal). Recombinant bacteria or protein can be administered alone, or can be co-administered or sequentially administered with other treatments or therapies, Forms of administration may include suspensions, syrups or elixirs, and preparations for parenteral, subcutaneous, intradermal, intramuscular or intravenous administration (e.g., injectable administration) such as sterile suspensions or emulsions. In one example, the recombinant bacteria or protein expressed by the method as defined herein is administered via the oral route.

For oral administration, the formulation of the recombinant bacteria or protein may be presented as capsules, tablets, powders, granules, or as a suspension. The preparation may have conventional additives, such as lactose, mannitol, corn starch, or potato starch. The preparation also may be presented with binders, such as crystalline cellulose, cellulose derivatives, acacia, corn starch, or gelatins. Additionally, the preparation may be presented with disintegrators, such as corn starch, potato starch, or sodium carboxymethylcellulose. The preparation may be further presented with dibasic calcium phosphate anhydrous or sodium starch glycolate. The preparation may be presented with lubricants, such as talc or magnesium stearate.

For intravenous, cutaneous, or subcutaneous injection, or injection at the site of affliction, the active ingredient will be in the form of a parenterally acceptable aqueous solution which is pyrogen-free and has suitable pH, isotonicity, and stability. Those of relevant skill in the art are well able to prepare suitable solutions using, for example, isotonic vehicles such as Sodium Chloride Injection, Ringer's Injection, or Lactated Ringer's Injection. Preservatives, stabilizers, buffers, antioxidants, and/or other additives can be included, as required.

For intranasal administration (e.g., nasal sprays) and/or pulmonary administration (administration by inhalation), formulations of the bacterial or protein preparation, including aerosol formulations, may be prepared in accordance with procedures well known to persons of skill in the art. Aerosol formulations may comprise either solid particles or solutions (aqueous or non-aqueous). Nebulizers (e.g., jet nebulizers, ultrasonic nebulizers, etc.) and atomizers may be used to produce aerosols from solutions e, g., using a solvent such as ethanol): metered-dose inhalers and dry-powder inhalers may be used to generate small-particle aerosols. The desired aerosol particle size can be obtained by employing any one of a number of methods known in the art, including, without limitation, jet-milling, spray drying, and critical-point condensation.

The term “treating” includes remedying a disease state or symptoms, preventing the establishment of disease, or otherwise preventing, hindering, retarding, or reversing the progression of disease or other undesirable symptoms in any way whatsoever. In one example, the disease is a gut disease, The gut disease may be a Celiac disease.

The term “patient” refers to patients of human or other mammal and includes any individual it is desired to examine or treat using the methods of the invention. However, it will be understood that “patient” does not imply that symptoms are present. Suitable mammals that fall within the scope of the invention include, but are not restricted to, primates, livestock animals (e.g. sheep, cows, horses, donkeys, pigs), laboratory test animals (e.g. rabbits, mice, rats, guinea pigs, hamsters), companion animals (e.g. cats, dogs) and captive wild animals (e.g. foxes, deers).

The recombinant lactic acid bacterium or the protein as expressed by the method as defined herein may be administered at a “pharmaceutically effective amount” to the patient in need thereof. The term “pharmaceutically effective amount” includes within its meaning a non-toxic but sufficient amount of an agent or compound to provide the desired therapeutic effect. The exact amount required will vary from subject to subject depending on factors such as the species being treated, the age and general condition of the subject, the severity of the condition being treated, the particular agent being administered and the mode of administration and so forth. Thus, it is not possible to specify an exact “pharmaceutically effective amount”. However, for any given case, an appropriate “pharmaceutically effective amount” may be determined by one of ordinary skill in the art using only routine experimentation.

In one example, there is provided a recombinant lactic acid bacterium as defined herein or a protein expressed by the method as defined herein for use in treating a gut disease.

In one example, there is provided a recombinant lactic acid bacterium as defined herein or a protein expressed by the method as defined herein when used in treating a gut disease.

In one example, there is provided the use of a recombinant lactic acid bacterium as defined herein or a protein expressed by the method as defined herein in the manufacture of a medicament for the treatment of a gut disease.

In one example, there is provided a kit comprising one or more compartments, wherein the kit comprises a first compartment adapted to contain a recombinant lactic acid bacterium as defined herein or a protein expressed by the method as defined herein. The kit may used to detect a patient suffering from a gut disease, such as a Celiac disease. The kit may also be used to detect the presence of excess gluten in a sample obtained from a patient suffering from a Celiac disease. In one example, the protein expressed by the method as defined herein cleaves gluten or a biomarker molecule. The biomarker molecule may be a biomarker molecule for Celiac disease. The kit may detect one or more products of the cleavage reaction. The recombinant bacteria may optionally be in freeze dried or other reconstitutable forms. The kit may comprise one or more other compartments adapted to contain one or more reagents.

### EXAMPLES

Aspects disclosed herein are further described by the following non-limiting Examples.

### Methods

### Bacteria, LAB Strains, Vector and Culture Media

NZ9000 strain and pNZ8148 plasmid of NICE [registered trademark] Expression System [a Lactococcus lactis expression vector employing a nisA promoter] were obtained from Boca Scientific. Genes were synthesized by Integrated DNA Technologies. Growth media, M17 and GM17, were obtained from BD Biosciences (USA).

Protein Cassette Construction on pNZ8148

Genes were codon-optimized using Integrated DNA Technologies' codon optimization tool for Lactococcus lactis cremoris. The codon-optimized genes were amplified from synthesized Gblocks [registered trademark] gene fragments (Integrated DNA Technologies) [double-stranded sequence-verified genomic blocks for gene construction] using the KOD-Xtreme kit (Merck). The PCR products were DpnI-treated for at least 2 hours and then cleaned up and concentrated using the DNA clean and concentrator kit (Zymo research). pNZ8148 were digested with restriction enzymes for at least 5 hours at 37° C. They were then incubated with thermosensitive alkaline phophatase TSAP (Promega) for 2 hours before they were cleaned and concentrated. The genes were then assembled into the vectors using Gibson assembly mix (New England Biolabs) for 1 hour at 50° C., 2 μL of the Gibson assembly mixture was added into 50 μL of electrocompetent NZ9000 cells and electioporated using 0.1 cm cuvette at 1800 V. 1 mL of GM17 media with 20 friM MgCl₂ and 2 mM CaCl₂ was added immediately after electroporation. The cuvette was kept on ice for 5 min before incubating the cells at 30° C. for 1 to 2 hours. The cells were centrifuged and resuspended in 100 μL of media before they were plated out on M17 with 0.5% glucose (GM17) agar with 10 μg./ml chloramphenicol and incubated at 30° C. for 2 days. Colonies were screened for the correct construct before isolation and sequencing of the plasmids were performed.

### Protein Expression and Cell Lysate Fraction Extraction

2% of overnight culture was inoculated into 50 mL of fresh GM17 media. The culture was grown statically at 30° C. to OD₆₀₀ 0.5 before inducing with 10 ng/mL of nisin. The culture, supernatant and cell pellet, was harvested 3 hours after nisin induction by centrifugation at 4600 rpm for 10 min. The cell pellet was washed and re-suspended in 300 μL of Lysis Equilibration Wash buffer (LEW buffer: 50 mM NaH₂PO₄, 300 mM NaCl, pH 8.0). 1 mg/mL lysozyme and 50 U/mL mutanolysin were added to the cell suspensions and the cell suspensions were incubated at 30° C. for 30 min. The cell suspensions were kept on ice and sonicated 4 tunes for 10 seconds at 10 seconds interval using Microsoft XL2000 sonicator at 22.5 kHz. The cell lysate was span down at 10,000 g for 30 min at 4° C. and the supernatant was removed as the soluble fraction. The remaining pellets were washed and re-suspended in denaturing buffer (50 mM NaH₂PO₄, 300 mM NaCl, 8M Urea, pH 8.0) and spun down (10,000 g, 20 min). to obtain the insoluble fraction. Biological triplicates were performed.

### Secretion Fraction

30 mL of the media fraction after 3 h of induction was removed. This was buffer exchanged and concentrated to 200 μL at 4° C. using a amicon ultra centrifudgal filter with cold 10 mM sodium phosphate buffer, pH 7.0. To ensure that cell lysis of NZ9000 strains was insignificant during production, the genomic DNA content in culture media of intact cells was compared to completely lysed cells at the point of harvest, using quantitative PCR with L. lactis specific primers for the housekeeping tuf gene (as described in Ruggirello et al, PloS one. 9(12) (2014): e114280). Based on this comparison, cell lysis was predicted to be kept to under 0.1%.

### Immunoblotting

Protein samples were analysed on NuPAGE 4-12% w/v or 12% w/v Bis-Tris Gel (Life Technologies). The proteins were then transferred on to a nitrocellulose membrane using semi-dry method (Trans-Blot; Biorad) at 20 V for 20 min. The membrane was washed with PBST (PBS with 0.1% v/v Tween) and then blocked using 5% w/v non-fat dry milk in PBST (Biorad) for 1 hour at room temperature and then washed with PBST. 1:10000 Anti-His Antibodies (Millipore) in Signal Enhancer HIKARI Solution B (Nacalai Tesque) was added to the membrane and incubated at 4° C. overnight before detecting with Clarity Western ECL Blotting Substrates (Biorad) using manufacturer's protocol. Densitometry was performed using ImageJ. Protein yields were calculated with respect to the no-propeptide-containing-USP45 construct, using secreted proteins per culture volume. Secretion efficiencies are calculated as the secreted protein over total proteins produced.

### Z-gly-pro-4-Nitroanilide Assay

2 μL of the concentrated secreted fraction was added to a mixture containing 5 μL of 2 mM Z-gly-pro-4-nitroanilide (in 1,4-dioxane) and 100 μL of 10 mM sodium phosphate buffer, pH 7.0. The release of p-nitroanilid.e was measured at 410 nm using technical triplicates. Enzymatic activity of secreted protein was also estimated based on a standard curve calibrated using purified FmPEP (Sigma- Aldrich).

### Example 1

### Secretion of TRX and FM PEP Proteins

### Mining of Propeptides

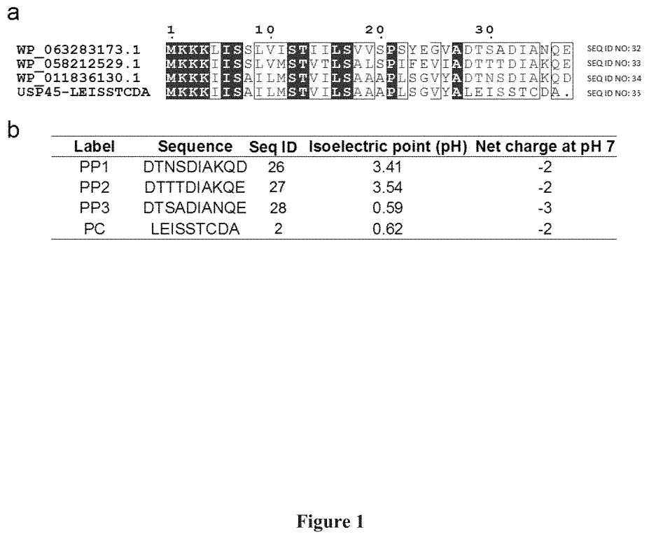

A reference signal peptide was first chosen, i.e. the naturally occurring signal peptide from L. lactis secreted protein of unknown function (USP45 SP). Besides being the currently most utilized secretion signal peptide for L. lactis, USP45 SP has also been shown to be more efficient over other natural signal peptides (SP310, Ravn, Peter, et al, Gene 242.1 (2000): 347-356) or mutated libraries (USP45MT11 Ng, Daphne T W et al, Applied and environmental microbiology 79.1 (2013): 347-356, SP310mut2, Ravn, Peter et al, Microbiology 149,8 (2003): 2193-2201). To mine for secretion propeptides, the amino acid sequence of ⁻USP45 SP (accession code: ABY84357) was used to blast against 109 deposited assemblies of Lactococcus species. From the BlastP search, three propeptide sequences, which were most representative of the results, were identified. (FIG. 1a). The isoelectric point of these propeptides ranged from 0.6 to 3.5, compared to 0.6 for our positive control propeptide LEISSTCDA (SEQ ID NO: 2). All three propeptides, has a net charge of −2 to −3 at pH 7 (FIG. 1b). The negative charges of the propeptides are contributed by the multiple aspartic acid and glutamic acid residues in the sequences. This observation also corresponds to the previous finding that negative charge of LEISSTCDA (SEQ ID NO: 2) plays a significant role in increasing secretion efficiencies. Hereforth for simplicity, the three data-mined propeptides will be labelled as PP1-PP3 (FIG. 1b).

### TRX Secretion and Optimization

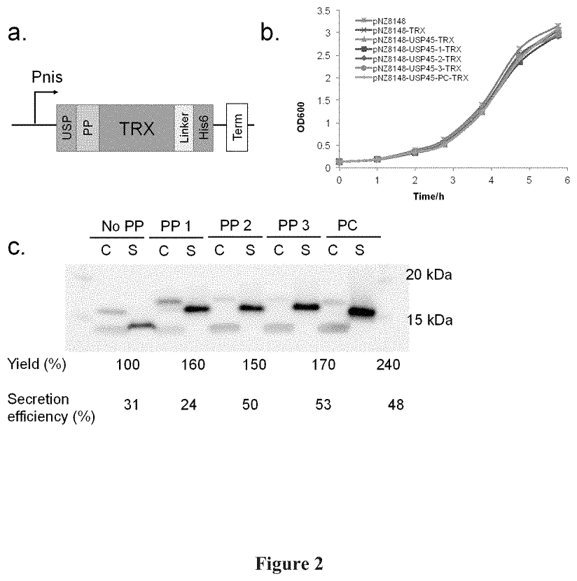

As an initial evaluation of these propeptides, the soluble 15 kDa E. coli TRX was used as a reporter protein. The glycine-serine linked protein expression cassette (FIG. 2a) consisted of USP45 SP, followed by the propeptide of interest, the N-terminal of the codon-optimized TRX gene cassette. The C-terminal of the gene cassette consisted of a glycine-serine-alanine (GSGSGAAA (SEQ ID NO: 29)) linker before a TEV cleavage site (ENLYFQG (SEQ ID NO: 30)) and a his6-tag (HHHHHH (SEQ ID NO: 31)), In the control expression cassette, there are no propeptide and only one GS linker between USP45 SP and TRX. The protein expression cassette was introduced into nisin-inducible pNZ8148 via DNA assembly.

The final constructs were transformed into NZ9000 and grown at 30° C. Induction of the culture proceed at OD600=0.5 with 10 ng/mL nisin (FIG. 2b). After 3 hours of expression, his-tagged proteins of the expected sizes (15-19 kDa) were observed (FIG. 2c). Bands on the Western blot, corresponding to full length (17 kDa) USP45-TRX and its truncated (15 kDa) TRX, were observed for USP45-TRX in intracellular and secreted fractions respectively.

Similarly, USP45-propeptide-TRX, bands corresponding to full length (19 kDa) USP45-propeptide TRX and truncated (16 kDa) propeptide-TRX were also observed in intracellular and extracellular fractions respectively. From the observed sizes of the TRX constructs, truncation of the secreted protein is predicted to occur between the signal peptide and propeptides. This is as predicted with SignalP (http://www.cbs.dtu.dk/services/SignalP, Petersen, Thomas Nordahl et al, Nature methods 8.10 (2011): 785-786).

With USP45 SP only, a 31% secretion efficiency for TRX was observed. Whilst upon insertion of propeptides, a maximum of 1.7 fold improvement in secretion efficiency (PP3, 53%) was observed. With increased secretion efficiencies, there are also corresponding 1.5-2.4 fold increases in secretion yields for propeptide containing constructs, compared to without propeptide (FIG. 2c). Overall, PP1-3 were observed to enhance both secretion efficiencies and yields like LEISSTCDA.

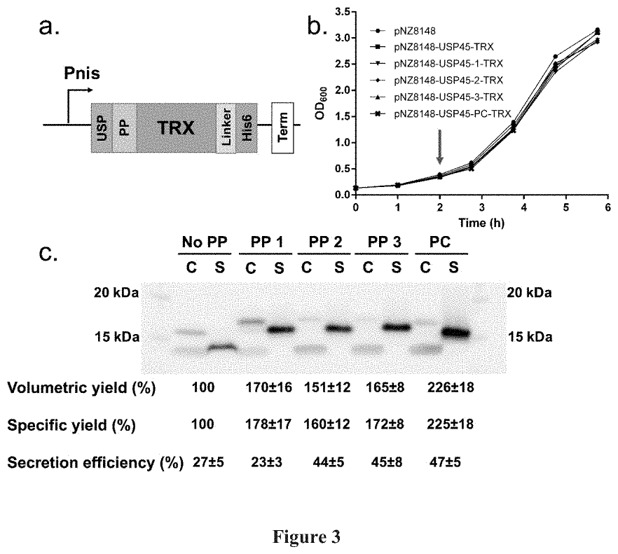

The experiment was repeated using the soluble 15 kDa E. coli TRX as the reporter protein (FIG. 3a). The final constructs were transformed into NZ9000 and grown at 30° C. Induction of the culture proceed at OD600=0.5 with 10 ng /mL nisin (FIG. 3b). After 3 hours of expression, his-tagged proteins of the expected sizes (15-19 kDa) were observed (FIG. 3c). Bands on the Western blot, corresponding to full length (17 kDa) USP45-TRX and its truncated (15 kDa) TRX, were observed for USP45-TRX in intracellular and secreted fractions respectively. Similarly, for USP45-propeptide-TRX constructs, bands corresponding to full length (19 kDa) USP45-propeptide TRX and truncated (16 kDa.) propeptide-TRX were also observed in intracellular and extracellular fractions respectively.

With USP45 SP only, a 27% secretion efficiency for TRX was observed. Whilst upon insertion of propeptides, a maximum of 1.7 fold improvement in secretion efficiency (LEISSTCDA, 47%, FIG. 3c) was observed. With increased secretion efficiencies, there are also corresponding 1.5-2.3 fold increases in both volumetric and specific secretion yields for propeptide containing constructs, compared to without propeptide (FIG. 3c). Overall, PP1-3 were observed to enhance both secretion efficiencies and yields like LEISSTCDA.

### Expression and Secretion of Functional Fm PEP

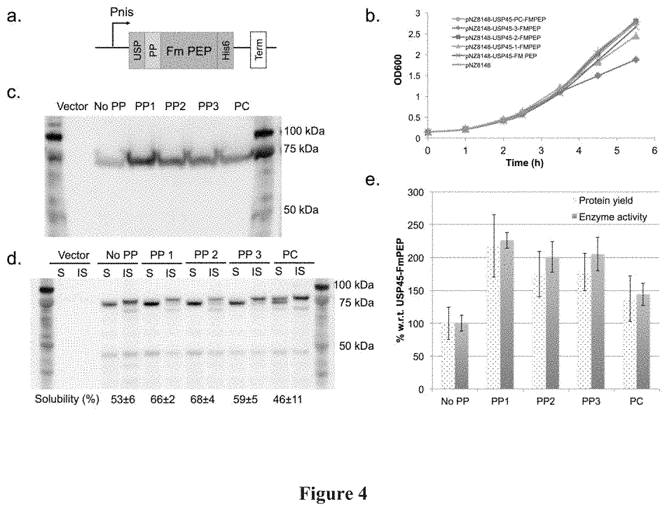

Next, the effects of PP1-3 on the secretion of Fm PEP were examined. The glycine-serine linked protein expression cassette consisted of USP45 SP, followed by the propeptide of interest, the codon-optimized Fm PEP gene cassette and a his6-tag at the C-terminal (FIG. 4a). In the control expression cassette, there are no propeptide and only one GS linker between USP45 SP and Fm PEP. Again, the protein expression cassette was introduced into nisin-inducible pNZ8148 via. DNA assembly.

After nisin induction of NZ9000-transformed strains at 30° C. for 3 hours (FIG. 4b), both cell lysate and media were analysed by Western blot analysis (FIG. 4c, d). Bands at 75 kDa corresponding to the size of Fm PEP were observed in both cell lysate and media fractions (FIG. 4c, d). Two closely migrated hands, observed near 75 kDa, are predicted to be full length constructs (84 and 83 kDa for with and without propeptide respectively) and truncated Fm PEP (81 and 80 kDa for with and without propeptide respectively). Bands of the truncated Fm PEPs were also observed in the soluble intracellular fraction, suggesting that the precursors are undergoing cleavage within the cells. In the intracellular fractions, solubility of the proteins range from 47-63% of the total intracellular proteins, and from our enzymatic assays, the soluble intracellular proteins were found to be active. A general increase in secretion yields (1.4 to 2.2 fold) was observed with the introduction of the propeptides into the Fm PEP expression cassette (FIG. 4e).

Comparison of PEP activities in the media fractions also displayed a similar enhancement trend to that of the secretion yields (FIG. 4e). This also suggests that there are minimal effects on Fm PEP activity from the different highly negative charged propeptides. Comparison between propeptides also indicated that a different preference of propeptide was demonstrated by Fm PEP compared to TRX. For Fm PEP, PP1 produced the best secretion yield and activity (2.2 fold) whilst the positive control LEISSTCDA (SEQ ID NO: 2) only managed a 1.4 fold increase in yield and activity.

Although secretion of active Fm PEP in L. lactis was demonstrated, the secretion efficiencies for Fm PEP (0.9-1.6%) is lower than that of TRX (24-53%). This could be attributed to the higher solubility and lower molecular weight of TRX compared to Fm PEP. Strikingly, although folding is not expected to take place with the presence of the signal peptide intracelfularly, the cell lysate is consisted of 46-69% soluble, cleaved Fm PEP proteins, wich are functional in our PEP assay (FIG. 4d). These observations suggest that further design optimization could be used to reduce intracellular cleavage and in turn, increase secretion efficiencies.

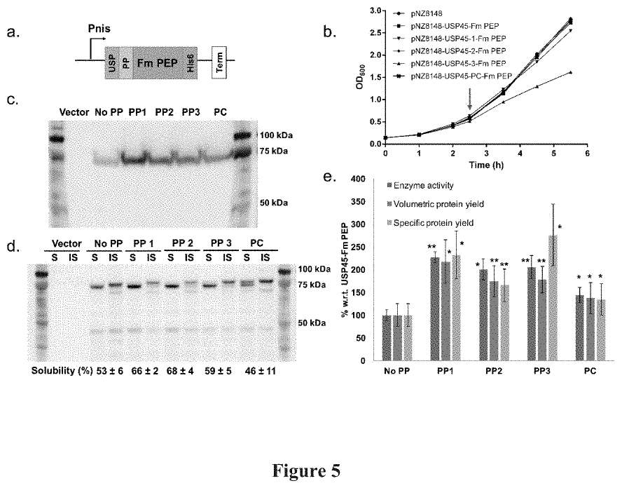

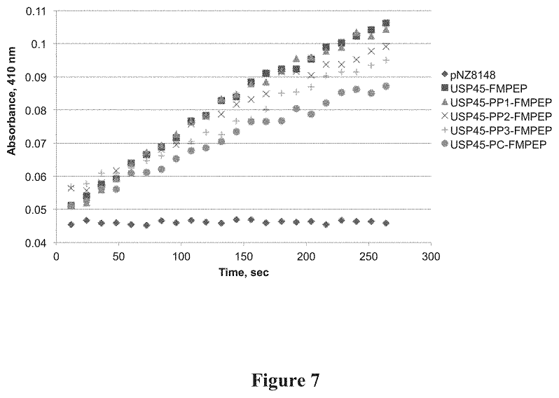

The experiment on the effects of PP1-3 on the secretion of Fm PEP Was repeated using the same expression cassette (FIG. 5a). After nisin induction of NZ9000-transformed strains at 30° C. for 3 hours (FIG. 5b), both cell lysate and media were analysed by Western blot analysis (FIG. 5c, d). Bands at 75 kDa corresponding to the size of Fm PEP were observed in both cell lysate and media fractions (FIG. 5c, d). Two closely migrated bands, observed near 75 kDa, are predicted to be full length constructs (84 and 83 kDa for with and without propeptide respectively) and truncated. Fm PEP (81 and 80 kDa for with and without propeptide respectively) (FIG. 5c, d). Bands of the truncated Fm PEPS were observed in the soluble intracellular fraction, suggesting that the precursors are undergoing cleavage within the cells. In the intracellular fractions, solubility of the proteins range from 46-68% of the total intracellular proteins, and from our enzymatic assays, the soluble intracellular proteins were found to be active (FIG. 7). A general increase in volumetric secretion yields (1.4 to 2.2 fold) was observed with the introduction of the propeptides into the Fm PEP expression cassette (FIG. 5e). When normalized to optical density, increases in specific secretion yields are 2.3. 1.7, 2.8 and 1.3 fold for PP1, PP2, PP3 and LEISSTCDA respectively (see Table 1). The higher specific yield of PP3 is a result of significant reduced growth of the host cell after nisin induction (FIG. 5b).

TABLE 1
Comparison of Fm PEP constructs

| | % w.r.t. to USP45-no PP- Fm PEP (s.d.) | | |
|---|---|---|---|
| | | Volumetric protein | Specific protein |
| | Enzyme activity | yield | yield |
| No PP | 100 (12) | 100 (25) | 100 (25) |
| PP1 | 226 (12) | 218 (47) | 232 (53) |
| PP2 | 201 (23) | 175 (34) | 166 (35) |
| PP3 | 205 (25) | 178 (29) | 276 (68)* |
| PC | 144 (17) | 137 (35) | 134 (34) |

*average OD of PP3 is 1.8 compared to 2.7 for no PP

Comparison of PEP activities in the media fractions also displayed a similar enhancement trend to that of the secretion yields (FIG. 5e). This also suggests that there are minimal effects on Fm PEP activity from insertion of different highly negative charged propeptides at the N-terminus. Comparison between propeptides also indicated that a different preference of propeptide was demonstrated by Fm PEP compared to TRX For Fm PEP, PP1 produced the best volumetric secretion yield and activity (2.2 fold) whilst the positive control LEISSTCDA (SEQ ID NO: 2) only managed a 1.4 fold increase in yield and activity.

As mentioned above, the lowed secretion efficiencies for Fm PEP as compared to that of TRX could possibly be attributed to the higher solubility and lower molecular weight of TRX compared to Fm PEP. Strikingly, although folding is not expected to take place with the presence of the signal peptide intracellularly, the cell lysate was found to contain soluble, cleaved and functional Fm PEP proteins (FIG. 5d, FIG. 7), These observations suggest that further design optimization could be used to reduce intracellular cleavage and in turn, increase secretion efficiencies

### Example 2

### Efficacy of the 3 Propeptides

In this study, 3 naturally occurring propeptides were examined in addition to the widely utilized synthetic propeptide LEISSTCDA (SEQ ID NO: 2). The set of 4 propeptides were evaluated using two different recombinant protein, where the ability to increase secretion yields and efficiencies were demonstrated for all 4 propeptides. However, from the subset of 2 proteins, it was shown that the optimal propeptide for each protein with USP45 SP is not the same. With TRX, the highest secretion efficiencies were obtained by PP3. With Fm PEP, the highest volumetric yield and secretion efficiencies were obtained by PP1.

From deposited genomics data, three new peptides have been identified for secretion enhancement. Through characterization of these three propeptides, along with a positive control LEISSTCDA, it was demonstrated that these propeptides are comparable to LEISSTCDA (SEQ ID NO: 2) as a secretion enhancement but also in the optimization of Fm PEP, they outperformed LEISSTCDA (SEQ ID NO: 2). Depending on the combination of protein of interest and propeptides, 1.4-2.3 fold increase in volumetric secretion yields were observed. In this work, it is demonstrated, for the first time, expression and secretion of functional Fm PEP in L. lactis.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

### REFERENCES

Ng, Daphne T W et al, Applied and environmental microbiology 79.1 (2013): 347-356

Petersen, Thomas Nordahl et al, Nature methods 8.10 (2011): 785-786

Ravn, Peter, et al, Gene 242.1 (2000): 347-356

Ravn, Peter et al, Microbiology 149.8 (2003): 2193-2201

Ruggirello M et al, PloS one. 9(12) (2014): e114280

1. An expression construct encoding a fusion protein, wherein the construct comprises
a) a first nucleic acid sequence encoding a peptide of general formula (I):
(SEQ ID NO 1)
X₁-X₂-X₃-X₄-X₅-X₆-X₇-X₈-X₉-X₁₀.
wherein each of X₁, X₅ and X₁₀ is a negatively charged amino acid or a functional variant thereof,
wherein each of X₂ , X₃, X₄, X₆, X₇ and X₉ is a polar or non-polar amino acid, or a functional variant thereof,
wherein X₈ is a positively-charged or a polar amino acid, or a functional variant thereof;
and
b) a second nucleic acid sequence encoding a protein for expression;
wherein the first nucleic acid sequence is contiguous to the second nucleic acid sequence , such that the peptide encoded by the first nucleic acid sequence and the protein encoded by the second nucleic acid sequence forms a fusion protein;
wherein the peptide encoded by the first nucleic acid sequence improves the expression of the protein encoded by the second nucleic acid sequence as compared to when the peptide encoded by the first nucleic acid sequence is absent.

2. The expression construct of claim 1, wherein:
i) each of X₁, and X₅ is an aspartic acid, or a functional variant thereof,
ii) X₂ is a threonine or a functional variant thereof,
iii) X₃ is selected from the group consisting of asparagine, threonine, and serine, or a functional variant thereof,
iv) X₄ is selected from the group consisting of serine, threonine and alanine, or a functional variant thereof,
v) X₆ is an isoleucine, or a functional variant thereof,
vi) X₇ is an alanine or a functional variant thereof,
vii) X₈ is selected from the group consisting of lysine and asparagine, or a functional variant thereof,
Viii) X₉ is a glutamine or a functional variant thereof, and
iX) X₁₀ is selected from the group consisting of aspartic acid and glutamic acid, or a functional variant thereof.

3. The expression construct of claim 1, wherein the peptide encoded by the first nucleic acid sequence is of general formula (Ia):
(SEQ ID NO: 25)
D-T-X₃-X₄-D-I-A-X₈-Q-X₁₀,
wherein:
i) X₃ is selected from the group consisting of asparagine, threonine, and serine, or a functional variant thereof,
ii) X₄ is selected from the group consisting of serine, threonine and alanine, or a functional variant thereof,
iii) X₈ is selected from the group consisting of lysine and asparagine, or a functional variant thereof, and
iv) X₁₀ is selected from the group consisting of an aspartic acid and a glutamic acid, or a functional variant thereof.

4. The expression construct of claim 1, wherein the peptide encoded by the first nucleic acid sequence is at least 60% identical to a sequence selected from the group consisting of:
a)
(SEQ ID NO: 26)
DTNSDIAKQD;
b)
(SEQ ID NO: 27)
DTTTDIAKQE;
and
c)
(SEQ ID NO: 28)
DTSADIANQE.

5. The expression construct of claim 1, wherein the peptide encoded by the first nucleic acid sequence has a net negative charge of −2 to −3 at pH 7.

6. The expression construct of claim 1, wherein the protein encoded by the second nucleic acid sequence is prolyl endopeptidase (PEP) or thioredoxin.

7. The expression construct of claim 6, wherein the prolyl endopeptidase (PEP) is selected from the group consisting of a Myxococcus Xanthus prolyl endopeptidase, a Flavobacterium meningosepticum prolyl endopeptidase, an Aspergillus niger prolyl endopeptidase and a Sphingomonas capsulate prolyl endopeptidase.

8. The expression construct of claim 1, wherein the peptide encoded by the first nucleic acid sequence is positioned between a signal peptide and the protein encoded by the second nucleic acid sequence.

9. A recombinant lactic acid bacterium comprising an expression construct of any one of claims 1-8.

10. The recombinant lactic acid bacterium of claim 9, wherein the genus of the lactic acid bacterium is selected from the group consisting of Lactobacillus, Lactococcus, Aerococcus, Leuconostoc, Oenococcus, Pediococcus, Streptococcus, Enterococcus, Weisella, Alloiococcus, Carnobacterium, Dolosigranulum, Globicatella, Tetragenococcus and Vagococcus.

11. The recombinant lactic acid bacterium of claim 10, wherein the lactic acid bacterium is Lactococcus lactis or Lactobacillus spp.

12. A method of expressing a protein in a lactic acid bacterium, the method comprising the steps of culturing the recombinant lactic acid bacterium of any one of claims 9-11 and isolating the protein expressed by the bacterium.

13. A recombinant lactic acid bacterium of any one of claims 9-11 or a protein expressed by the method of (12) for use as a medicament.

14. A method of treating a gut disease, the method comprising the step of administering a recombinant lactic acid bacterium of any one of claims 9-11 or a protein expressed by the method of claim 12 to a patient in need thereof.

15. The method of claim 14, wherein the gut disease is a Celiac disease.

16. A recombinant lactic acid bacterium of any one of claims 9-11 or a protein expressed by the method of claim 12 for use in treating a gut disease.

17. The recombinant lactic acid bacterium or the protein of claim 16, wherein the gut disease is Celiac disease.

18. Use of a recombinant lactic acid bacterium of any one of claims 9-11 or a protein expressed by the method of claim 12 in the manufacture of a medicament for the treatment of a gut disease.

19. The use of claim 18, wherein the gut disease is Celiac disease.


SEQUENCE LISTING (35 sequences)

SEQ ID NO: 1 - protein, 10 aa, Artificial Sequence
  Designed peptide
  misc_feature (1)..(1): Xaa is a negatively charged amino acid or a functional variant thereof.
  misc_feature (2)..(4): Xaa is a polar or non-polar amino acid or a functional variant thereof.
  misc_feature (5)..(5): Xaa is a negatively charged amino acid or a functional variant thereof.
  misc_feature (6)..(7): Xaa is a polar or non-polar amino acid or a functional variant thereof.
  misc_feature (8)..(8): Xaa is a positively-charged or a polar amino acid, or a functional variant thereof.
  misc_feature (9)..(9): Xaa is a polar or non-polar amino acid or a functional variant thereof.
  misc_feature (10)..(10): Xaa is a negatively charged amino acid or a functional variant thereof.
XXXXXXXXXX

SEQ ID NO: 2 - protein, 9 aa, Artificial Sequence
  Control
LEISSTCDA

SEQ ID NO: 3 - protein, 10 aa, Artificial Sequence
  Designed peptide
  misc_feature (2)..(4): Xaa is a polar or non-polar amino acid or a functional variant thereof.
  misc_feature (5)..(5): Xaa is a negatively charged amino acid or a functional variant thereof
  misc_feature (6)..(7): Xaa is a polar or non-polar amino acid or a functional variant thereof.
  misc_feature (8)..(8): Xaa is a positively-charged or a polar amino acid, or a functional variant thereof.
  misc_feature (9)..(9): Xaa is a polar or non-polar amino acid or a functional variant thereof.
  misc_feature (10)..(10): Xaa is a negatively charged amino acid or a functional variant thereof.
DXXXXXXXXX

SEQ ID NO: 4 - protein, 10 aa, Artificial Sequence
  Designed peptide
  misc_feature (1)..(1): Xaa is a negatively charged amino acid or a functional variant thereof.
  misc_feature (3)..(4): Xaa is a polar or non-polar amino acid or a functional variant thereof.
  misc_feature (5)..(5): Xaa is a negatively charged amino acid or a functional variant thereof
  misc_feature (6)..(7): Xaa is a polar or non-polar amino acid or a functional variant thereof.
  misc_feature (8)..(8): Xaa is a positively-charged or a polar amino acid, or a functional variant thereof.
  misc_feature (9)..(9): Xaa is a polar or non-polar amino acid or a functional variant thereof.
  misc_feature (10)..(10): Xaa is a negatively charged amino acid or a functional variant thereof.
XTXXXXXXXX

SEQ ID NO: 5 - protein, 10 aa, Artificial Sequence
  Designed peptide
  misc_feature (1)..(1): Xaa is a negatively charged amino acid or a functional variant thereof.
  misc_feature (2)..(4): Xaa is a polar or non-polar amino acid or a functional variant thereof.
  misc_feature (6)..(7): Xaa is a polar or non-polar amino acid or a functional variant thereof.
  misc_feature (8)..(8): Xaa is a positively-charged or a polar amino acid, or a functional variant thereof.
  misc_feature (9)..(9): Xaa is a polar or non-polar amino acid or a functional variant thereof.
  misc_feature (10)..(10): Xaa is a negatively charged amino acid or a functional variant thereof.
XXXXDXXXXX

SEQ ID NO: 6 - protein, 10 aa, Artificial Sequence
  Designed peptide
  misc_feature (1)..(1): Xaa is a negatively charged amino acid or a functional variant thereof.
  misc_feature (2)..(4): Xaa is a polar or non-polar amino acid or a functional variant thereof.
  misc_feature (5)..(5): Xaa is a negatively charged amino acid or a functional variant thereof
  misc_feature (7)..(7): Xaa is a polar or non-polar amino acid or a functional variant thereof.
  misc_feature (8)..(8): Xaa is a positively-charged or a polar amino acid, or a functional variant thereof.
  misc_feature (9)..(9): Xaa is a polar or non-polar amino acid or a functional variant thereof.
  misc_feature (10)..(10): Xaa is a negatively charged amino acid or a functional variant thereof.
XXXXXIXXXX

SEQ ID NO: 7 - protein, 10 aa, Artificial Sequence
  Designed peptide
  misc_feature (1)..(1): Xaa is a negatively charged amino acid or a functional variant thereof.
  misc_feature (2)..(4): Xaa is a polar or non-polar amino acid or a functional variant thereof.
  misc_feature (5)..(5): Xaa is a negatively charged amino acid or a functional variant thereof
  misc_feature (6)..(6): Xaa is a polar or non-polar amino acid or a functional variant thereof.
  misc_feature (8)..(8): Xaa is a positively-charged or a polar amino acid, or a functional variant thereof.
  misc_feature (9)..(9): Xaa is a polar or non-polar amino acid or a functional variant thereof.
  misc_feature (10)..(10): Xaa is a negatively charged amino acid or a functional variant thereof.
XXXXXXAXXX

SEQ ID NO: 8 - protein, 10 aa, Artificial Sequence
  Designed peptide
  misc_feature (1)..(1): Xaa is a negatively charged amino acid or a functional variant thereof.
  misc_feature (2)..(4): Xaa is a polar or non-polar amino acid or a functional variant thereof.
  misc_feature (5)..(5): Xaa is a negatively charged amino acid or a functional variant thereof.
  misc_feature (6)..(7): Xaa is a polar or non-polar amino acid or a functional variant thereof.
  misc_feature (8)..(8): Xaa is a positively-charged or a polar amino acid, or a functional variant thereof.
  misc_feature (10)..(10): Xaa is a negatively charged amino acid or a functional variant thereof.
XXXXXXXXQX

SEQ ID NO: 9 - protein, 10 aa, Artificial Sequence
  Designed sequence
  misc_feature (3)..(4): Xaa is a polar or non-polar amino acid or a functional variant thereof.
  misc_feature (5)..(5): Xaa is a negatively charged amino acid or a functional variant thereof.
  misc_feature (6)..(7): Xaa is a polar or non-polar amino acid or a functional variant thereof.
  misc_feature (8)..(8): Xaa is a positively-charged or a polar amino acid, or a functional variant thereof.
  misc_feature (9)..(9): Xaa is a polar or non-polar amino acid or a functional variant thereof.
  misc_feature (10)..(10): Xaa is a negatively charged amino acid or a functional variant thereof.
DTXXXXXXXX

SEQ ID NO: 10 - protein, 10 aa, Artificial Sequence
  Designed peptide
  misc_feature (2)..(4): Xaa is a polar or non-polar amino acid or a functional variant thereof.
  misc_feature (6)..(7): Xaa is a polar or non-polar amino acid or a functional variant thereof.
  misc_feature (8)..(8): Xaa is a positively-charged or a polar amino acid, or a functional variant thereof.
  misc_feature (9)..(9): Xaa is a polar or non-polar amino acid or a functional variant thereof.
  misc_feature (10)..(10): Xaa is a negatively charged amino acid or a functional variant thereof.
DXXXDXXXXX

SEQ ID NO: 11 - protein, 10 aa, Artificial Sequence
  Designed peptide
  misc_feature (2)..(4): Xaa is a polar or non-polar amino acid or a functional variant thereof.
  misc_feature (5)..(5): Xaa is a negatively charged amino acid or a functional variant thereof.
  misc_feature (7)..(7): Xaa is a polar or non-polar amino acid or a functional variant thereof.
  misc_feature (8)..(8): Xaa is a positively-charged or a polar amino acid, or a functional variant thereof.
  misc_feature (9)..(9): Xaa is a polar or non-polar amino acid or a functional variant thereof.
  misc_feature (10)..(10): Xaa is a negatively charged amino acid or a functional variant thereof.
DXXXXIXXXX

SEQ ID NO: 12 - protein, 10 aa, Artificial Sequence
  Designed peptide
  misc_feature (2)..(4): Xaa is a polar or non-polar amino acid or a functional variant thereof.
  misc_feature (5)..(5): Xaa is a negatively charged amino acid or a functional variant thereof.
  misc_feature (6)..(6): Xaa is a polar or non-polar amino acid or a functional variant thereof.
  misc_feature (8)..(8): Xaa is a positively-charged or a polar amino acid, or a functional variant thereof.
  misc_feature (9)..(9): Xaa is a polar or non-polar amino acid or a functional variant thereof.
  misc_feature (10)..(10): Xaa is a negatively charged amino acid or a functional variant thereof.
DXXXXXAXXX

SEQ ID NO: 13 - protein, 10 aa, Artificial Sequence
  Designed peptide
  misc_feature (2)..(4): Xaa is a polar or non-polar amino acid or a functional variant thereof.
  misc_feature (5)..(5): Xaa is a negatively charged amino acid or a functional variant thereof.
  misc_feature (6)..(7): Xaa is a polar or non-polar amino acid or a functional variant thereof.
  misc_feature (8)..(8): Xaa is a positively-charged or a polar amino acid, or a functional variant thereof.
  misc_feature (10)..(10): Xaa is a negatively charged amino acid or a functional variant thereof.
DXXXXXXXQX

SEQ ID NO: 14 - protein, 10 aa, Artificial Sequence
  Designed peptide
  misc_feature (1)..(1): Xaa is a negatively charged amino acid or a functional variant thereof.
  misc_feature (3)..(4): Xaa is a polar or non-polar amino acid or a functional variant thereof.
  misc_feature (6)..(7): Xaa is a polar or non-polar amino acid or a functional variant thereof.
  misc_feature (8)..(8): Xaa is a positively-charged or a polar amino acid, or a functional variant thereof.
  misc_feature (9)..(9): Xaa is a polar or non-polar amino acid or a functional variant thereof.
  misc_feature (10)..(10): Xaa is a negatively charged amino acid or a functional variant thereof.
XTXXDXXXXX

SEQ ID NO: 15 - protein, 10 aa, Artificial Sequence
  Designed peptide
  misc_feature (1)..(1): Xaa is a negatively charged amino acid or a functional variant thereof.
  misc_feature (3)..(4): Xaa is a polar or non-polar amino acid or a functional variant thereof.
  misc_feature (5)..(5): Xaa is a negatively charged amino acid or a functional variant thereof.
  misc_feature (7)..(7): Xaa is a polar or non-polar amino acid or a functional variant thereof.
  misc_feature (8)..(8): Xaa is a positively-charged or a polar amino acid, or a functional variant thereof.
  misc_feature (9)..(9): Xaa is a polar or non-polar amino acid or a functional variant thereof.
  misc_feature (10)..(10): Xaa is a negatively charged amino acid or a functional variant thereof.
XTXXXIXXXX

SEQ ID NO: 16 - protein, 10 aa, Artificial Sequence
  Designed peptide
  misc_feature (1)..(1): Xaa is a negatively charged amino acid or a functional variant thereof.
  misc_feature (3)..(4): Xaa is a polar or non-polar amino acid or a functional variant thereof.
  misc_feature (5)..(5): Xaa is a negatively charged amino acid or a functional variant thereof.
  misc_feature (6)..(6): Xaa is a polar or non-polar amino acid or a functional variant thereof.
  misc_feature (8)..(8): Xaa is a positively-charged or a polar amino acid, or a functional variant thereof.
  misc_feature (9)..(9): Xaa is a polar or non-polar amino acid or a functional variant thereof.
  misc_feature (10)..(10): Xaa is a negatively charged amino acid or a functional variant thereof.
XTXXXXAXXX

SEQ ID NO: 17 - protein, 10 aa, Artificial Sequence
  Designed peptide
  misc_feature (1)..(1): Xaa is a negatively charged amino acid or a functional variant thereof.
  misc_feature (3)..(4): Xaa is a polar or non-polar amino acid or a functional variant thereof.
  misc_feature (5)..(5): Xaa is a negatively charged amino acid or a functional variant thereof.
  misc_feature (6)..(7): Xaa is a polar or non-polar amino acid or a functional variant thereof.
  misc_feature (8)..(8): Xaa is a positively-charged or a polar amino acid, or a functional variant thereof.
  misc_feature (10)..(10): Xaa is a positively-charged or a polar amino acid, or a functional variant thereof.
XTXXXXXXQX

SEQ ID NO: 18 - protein, 10 aa, Artificial Sequence
  Designed peptide
  misc_feature (1)..(1): Xaa is a negatively charged amino acid or a functional variant thereof.
  misc_feature (2)..(4): Xaa is a polar or non-polar amino acid or a functional variant thereof.
  misc_feature (7)..(7): Xaa is a polar or non-polar amino acid or a functional variant thereof.
  misc_feature (8)..(8): Xaa is a positively-charged or a polar amino acid, or a functional variant thereof.
  misc_feature (9)..(9): Xaa is a polar or non-polar amino acid or a functional variant thereof.
  misc_feature (10)..(10): Xaa is a negatively charged amino acid or a functional variant thereof.
XXXXDIXXXX

SEQ ID NO: 19 - protein, 10 aa, Artificial Sequence
  Designed peptide
  misc_feature (1)..(1): Xaa is a negatively charged amino acid or a functional variant thereof.
  misc_feature (2)..(4): Xaa is a polar or non-polar amino acid or a functional variant thereof.
  misc_feature (6)..(6): Xaa is a polar or non-polar amino acid or a functional variant thereof.
  misc_feature (8)..(8): Xaa is a positively-charged or a polar amino acid, or a functional variant thereof.
  misc_feature (9)..(9): Xaa is a polar or non-polar amino acid or a functional variant thereof.
  misc_feature (10)..(10): Xaa is a negatively charged amino acid or a functional variant thereof.
XXXXDXAXXX

SEQ ID NO: 20 - protein, 10 aa, Artificial Sequence
  Designed peptide
  misc_feature (1)..(1): Xaa is a negatively charged amino acid or a functional variant thereof.
  misc_feature (2)..(4): Xaa is a polar or non-polar amino acid or a functional variant thereof.
  misc_feature (6)..(7): Xaa is a polar or non-polar amino acid or a functional variant thereof.
  misc_feature (8)..(8): Xaa is a positively-charged or a polar amino acid, or a functional variant thereof.
  misc_feature (10)..(10): Xaa is a negatively charged amino acid or a functional variant thereof.
XXXXDXXXQX

SEQ ID NO: 21 - protein, 10 aa, Artificial Sequence
  Designed peptide
  misc_feature (1)..(1): Xaa is a negatively charged amino acid or a functional variant thereof.
  misc_feature (2)..(4): Xaa is a polar or non-polar amino acid or a functional variant thereof.
  misc_feature (5)..(5): Xaa is a negatively charged amino acid or a functional variant thereof.
  misc_feature (8)..(8): Xaa is a positively-charged or a polar amino acid, or a functional variant thereof.
  misc_feature (9)..(9): Xaa is a polar or non-polar amino acid or a functional variant thereof.
  misc_feature (10)..(10): Xaa is a negatively charged amino acid or a functional variant thereof.
XXXXXIAXXX

SEQ ID NO: 22 - protein, 10 aa, Artificial Sequence
  Designed peptide
  misc_feature (1)..(1): Xaa is a negatively charged amino acid or a functional variant thereof.
  misc_feature (2)..(4): Xaa is a polar or non-polar amino acid or a functional variant thereof.
  misc_feature (5)..(5): Xaa is a negatively charged amino acid or a functional variant thereof.
  misc_feature (7)..(7): Xaa is a polar or non-polar amino acid or a functional variant thereof.
  misc_feature (8)..(8): Xaa is a positively-charged or a polar amino acid, or a functional variant thereof.
  misc_feature (10)..(10): Xaa is a negatively charged amino acid or a functional variant thereof.
XXXXXIXXQX

SEQ ID NO: 23 - protein, 10 aa, Artificial Sequence
  Designed peptide
  misc_feature (1)..(1): Xaa is a negatively charged amino acid or a functional variant thereof.
  misc_feature (2)..(4): Xaa is a polar or non-polar amino acid or a functional variant thereof.
  misc_feature (5)..(5): Xaa is a negatively charged amino acid or a functional variant thereof.
  misc_feature (6)..(6): Xaa is a polar or non-polar amino acid or a functional variant thereof.
  misc_feature (8)..(8): Xaa is a positively-charged or a polar amino acid, or a functional variant thereof.
  misc_feature (10)..(10): Xaa is a negatively charged amino acid or a functional variant thereof.
XXXXXXAXQX

SEQ ID NO: 24 - protein, 10 aa, Artificial Sequence
  Designed peptide
  misc_feature (3)..(4): Xaa is a polar or non-polar amino acid or a functional variant thereof.
  misc_feature (8)..(8): Xaa is a positively-charged or a polar amino acid, or a functional variant thereof.
  misc_feature (10)..(10): Xaa is a negatively charged amino acid or a functional variant thereof.
DTXXDIAXQX

SEQ ID NO: 25 - protein, 10 aa, Artificial Sequence
  Designed peptide
  misc_feature (3)..(3): Xaa is asparagine, threonine, or serine, or a functional variant thereof
  misc_feature (4)..(4): Xaa is serine, threonine or alanine, or a functional variant thereof
  misc_feature (8)..(8): Xaa is lysine and asparagine, or a functional variant thereof
  misc_feature (10)..(10): Xaa is aspartic acid and a glutamic acid, or a functional variant thereof
DTXXDIAXQX

SEQ ID NO: 26 - protein, 10 aa, Artificial Sequence
  Designed peptide
DTNSDIAKQD

SEQ ID NO: 27 - protein, 10 aa, Artificial Sequence
  Designed peptide
DTTTDIAKQE

SEQ ID NO: 28 - protein, 10 aa, Artificial Sequence
  Designed peptide
DTSADIANQE

SEQ ID NO: 29 - protein, 8 aa, Artificial Sequence
  Designed sequence
GSGSGAAA

SEQ ID NO: 30 - protein, 7 aa, Artificial Sequence
  Desgined sequence
ENLYFQG

SEQ ID NO: 31 - protein, 6 aa, Artificial Sequence
  Designed sequence
HHHHHH

SEQ ID NO: 32 - protein, 37 aa, Lactoccocus
MKKKLISSLVISTIILSVVSPSYEGVADTSADIANQE

SEQ ID NO: 33 - protein, 37 aa, Lactoccocus
MKKKIISSLVMSTVTLSALSPIFEVIADTTTDIAKQE

SEQ ID NO: 34 - protein, 37 aa, Lactoccocus
MKKKIISAILMSTVILSAAAPLSGVYADTNSDIAKQD

SEQ ID NO: 35 - protein, 36 aa, Lactococcus lactis
MKKKIISAILMSTVILSAAAPLSGVYALEISSTCDA